(19) United States
(12) Patent Application Publication
(10) Pub. No.: US 2003/0175254 A1
(43) Pub. Date: Sep. 18, 2003

(54) Method of obtaining purified enterochromaffin cells and methods of using same

(76) Inventors: George Sachs, Encino, CA (US); Irvin Modlin, Woodbridge, CT (US); Patrick Bruggeman, Palm Beach, FL (US)

Correspondence Address: GRAY CARY WARE & FREIDENRICH LLP, 4365 EXECUTIVE DRIVE, SUITE 1100, SAN DIEGO, CA, 92121-2133, US

(21) Appl. No.: 10/329,323

(22) Filed: Dec. 23, 2002

Related U.S. Application Data
(60) Provisional application No. 60/342,958, filed on Dec. 21, 2001.

(51) Int. Cl. C12Q 1/68; A61K 48/00; C12N 5/08

(52) U.S. Cl. 424/93.21; 435/6; 435/366

(57) ABSTRACT

Substantially purified enterochromaffin (EC) cells and methods of using the cells to identify nucleic acid sequences involved in disorders associated with enterochromaffin cells are provided. In addition, methods of treating enterochromaffin cell-associated disorders such as carcinoid tumor, carcinoid syndrome and irritable bowel disorder by targeting the identified nucleic acid sequences are provided.

### RELATED APPLICATION DATA

This application claims the benefit of priority under 35 USC 119(e)(1) of U.S. Ser. No. 60/342,958, filed Dec. 21, 2001, the entire content of which is incorporated herein by reference.

### FIELD OF THE INVENTION

The present invention relates to enterochromaffin (EC) cells and methods of using the cells to identify nucleic acid sequences involved in disorders associated with EC cells. The invention also relates to methods of treating EC cell-associated disorders such as carcinoid tumors, carcinoid syndrome and irritable bowel disorder.

### BACKGROUND OF THE INVENTION

Carcinoid tumors are the most protean and the most common gastrointestinal endocrine tumors, accounting for approximately 55 percent of such neoplasms. The incidence of carcinoid tumors in the United States is approximately 15 per 1 million people per year. Symptoms associated with carcinoid tumors include gastrointestinal bleeding, abdominal pain, gastrointestinal obstruction from tumor growth or tumor-induced mesenteric fibrosis or symptoms arising from tumor-secreted hormones. The name carcinoid was applied to these tumors because slow growth and the homogenous appearance of tumor cells led early investigators to underestimate their malignant potential. Typically, the interval between the onset of symptoms and diagnosis is about 4 to 5 years.

Carcinoid tumors arise from neuroendocrine cells throughout the body, but are most prevalent in the gastrointestinal tract, pancreas and pulmonary bronchi. Ninety percent of carcinoid tumors arise from EC cells in the gastrointestinal tract. The tumors can be found anywhere from the stomach to the rectum and are most common in the appendix, ileum and rectum. Gastrointestinal carcinoids frequently cause abdominal bleeding, or intestinal obstruction. Although the tumors are rarely large, they can become the leading point for intussusception, a serious problem with the intestine or bowel wherein part of the intestine collapses into itself with a loss of function. In addition, mesenteric spread stimulates a local fibrous reaction. No risk factors have been clearly defined for these tumors. Appendiceal tumors comprise nearly half of all carcinoid tumors and are usually small, solitary and benign. Colorectal carcinoids have a similarly benign course and are usually asymptomatic.

EC cells secrete a variety of hormones and are embryologically related to thyroid C cells, adrenal medullary cells, and melanocytes. Tumors of these cell types can produce syndromes of hormone excess. Hormone secretion by carcinoid cells can cause distinctive and debilitating effects, known as carcinoid syndrome, before local growth or metastatic spread is otherwise apparent.

Carcinoid tumors can be classed on the basis of embryonic origin. Clinical features, secreted hormones, diagnostic evaluation, and prognosis vary according to whether a carcinoid arises in EC cells of foregut, midgut, or hindgut structures. A variety of treatments are available to control the symptoms of carcinoid disease, although since most patients with the disease have metastatic disease, complete reversal, by surgery, for example, is rare. Modulation of EC cells to inhibit hormone secretion by carcinoid tumors is possible using certain therapeutic agents, however, relief of some symptoms is associated with serious side effects and certain symptoms are not controlled at all.

### SUMMARY OF THE INVENTION

The present invention is based upon the a method of identification of nucleic acids involved in disorders associated with EC cells. The invention also presents a method of isolation of EC cells. Additionally, the invention is based on methods of treating EC cell-associated disorders.

In one embodiment, the invention provides a method of preparing substantially purified EC cells. The method includes obtaining a mixed population of cells containing EC cells from a subject and separating the population of cells by a separation technique based on density of the cells, size of the cells, or both. Such separation techniques can include density gradient centrifugation and/or elutriation. After performance of the separation technique, a separated population containing EC cells is present. The invention further provides removing the EC cells from the separated population to obtain substantially purified EC cells.

In another embodiment, the invention provides a method of obtaining an isolated EC cell by contacting a population of cells containing EC cells with a reagent that specifically binds EC cells. After contacting with the reagent, any EC cells in the population are isolated. In an additional embodiment, the cells specifically bound by the reagent are detected prior to isolation. In the method of the invention, detection methods can include an immunocytochemical method, a histochemical method or in situ hybridization.

The invention also provides a method of identifying a polynucleotide associated with an enterochromaffin (EC) cell-associated disorder. The method is performed by contacting an EC cell from a subject having or suspected of having an EC cell-associated disorder with an array of probes representative of EC cell nucleic acid molecules expressed in EC cells from a subject having an EC cell-associated disorder. Expression of nucleic acid molecules in the EC cell with an EC cell-associated disorder is detected. Further, where the expression of nucleic acid molecules in the EC cell with an EC cell-associated disorder differs from the expression of nucleic acid molecules in a normal EC cell without an EC cell-associated disorder a polynucleotide associated with an EC cell-associated disorder in an EC cell is identified. In one embodiment of the invention, the EC cell-associated disorder is abnormal neuroendocrine hormone expression of an EC cell. In another embodiment, the EC cell-associated disorder is an EC cell proliferative disorder.

In still another embodiment, the invention provides a method of identifying a polynucleotide associated with enterochromaffin (EC) cell proliferation. The method is performed by contacting a test EC cell with an array of probes representative of EC cell nucleic acid molecules expressed in normal EC cells and detecting expression of nucleic acid molecules in the test EC cell. Additionally, a clustering analysis of the nucleic acid molecules expressed is performed. A cluster comprising polynucleotides associated with proliferation of EC cells is then detected, which allows identification of a polynucleotide associated with EC cell proliferation. In one embodiment, the polynucleotide is isolated. The invention also provides the isolated polynucleotide.

In still another aspect, the invention provides a method of treatment or prevention of an EC cell-associated disorder. The treatment method includes administering to a subject an agent that modulates expression of a polynucleotide associated with an EC cell-associated disorder. The administration is made to a subject having or suspected of having an EC cell-associated disorder.

In yet another aspect the invention provides a composition containing an antisense polynucleotide that specifically binds a mRNA encoding a polynucleotide associated with an EC cell-associated disorder and a carrier. In one embodiment the composition is a pharmaceutical composition.

The invention further provides a cDNA library prepared from EC cells obtained, as described above, from a subject having a EC cell-associated disorder, wherein the EC cells exhibit altered phenotypic expression.

In still another embodiment the invention provides a method of treating or preventing fibrosis. In the method, an agent that modulates expression of a factor essential for the production of fibrosis is administered to a subject having or suspected of having fibrosis.

### DETAILED DESCRIPTION OF THE INVENTION

The present invention provides methods of detecting and isolating enterochromaffin (EC) cells, and methods of using the isolated cells. EC cells are neuroendocrine cells that secrete a wide variety of hormones (peptidergic and non-peptidergic) and are embryologically related to thyroid C cells, melanocytes and adrenal medullary cells. Several subsets of EC cells have been identified and classified by their site of origin. EC cells can originate from or be found in the stomach, small intestine, pancreas, colon and appendix. Therefore, EC cells can be gastric, duodenal or intestinal in origin. EC cells can also be classified by the major hormone secreted. For example, EC cells secrete serotonin (5-hydroxytryptamine; 5-HT) and histamine, and peptides such as vasoactive intestinal polypeptide (VIP), glucagon, somatostatin, neurotensin, gastrin, guanylin and grhelin.

Concentrated in the EC cells of the mucosa of the mammalian intestine are large amounts of serotonin. EC cells have the enzymes to synthesize 5-HT, are endowed with a specific, imipramine-sensitive 5-HT uptake mechanism and can store 5-HT in specific secretory vesicles. EC cells can secrete 5-HT in a calcium-dependent manner. In particular, calcium influx through voltage-regulated channels and receptor-mediated liberation of intracellular calcium can evoke 5-HT release. 5-HT secretion from EC cells occurs predominantly at the interstitial side and is controlled by a complex pattern of receptor-mediated mechanisms. Stimulatory receptors (for example, beta-adrenoceptors, muscarinic, nicotine and 5-HT3 receptors) and inhibitory receptors (for example, alpha 2-adrenoceptors, histamine H3, GABAA and GABAB, A2 and P2Y alpha purine and 5-HT4 receptors as well as receptors for vasoactive intestinal polypeptide (VIP), pituitary adenylate cyclase stimulating peptide (PACAP) and somatostatin) can be involved in the control of 5-HT release from the EC cells.

The present invention provides a method of obtaining a population of substantially purified EC cells comprising density gradient centrifugation, elutriation or a combination thereof. As used herein, “substantially purified” refers to EC cells that have been separated from other cell types in a mixed population of cells. A population of substantially purified EC cells is a population that is at least about 75%, at least about 80%, at least about 85%, at least about 90%, at least about 95% or at least about 100% EC cells. In one aspect, a population of substantially purified EC cells is at least about 80% EC cells.

Density gradient separation can be used to purify cells. The most commonly used methods of separating mixed populations to obtain pure suspensions of a cell type make use of one or more of the centrifugation techniques known to those in the art. An important factor in obtaining a viable cell preparation by centrifugation is the choice of the gradient medium. Centrifugation can be used to separate cells on the basis of either size (for example, by elutriation) or buoyant density. When particles in a suspension are subjected to the centrifugal field, they move in the direction in which the force is applied. In general, larger particles will move (sediment) faster than smaller, providing an opportunity to separate large particles from small during centrifugation. As intact cells are relatively large particles, which will sediment rapidly at low centrifugal forces, a partial degree of purification can be obtained by low speed centrifugation in a salt or nutrient solution.

Elutriation can be used to separate cells on the basis of size. Centripetal flow of a solution over a sample of cells balances movement of the cells centrifugally so that cells that are larger are separated from cells that are smaller. A specific elutriation rotor speed is balanced by a specific flow rate of the solution to optimize size separation.

Cells can also be separated on the basis of their buoyant density. Some of the major compartments that contribute to the overall density of a cell are the nucleus, the aqueous cytosol, secretion granules and vacuoles, lipid droplets and the cytoskeleton. There are significant quantitative differences in these compartments in different cell types.

In order to purify a particular cell population on the basis of its buoyant density, it can be useful to first obtain a population partially purified by differential centrifugation, as described above. The initial purification will allow more of the cell population to be purified to be applied to the gradient without overloading it. Additionally, artefactual banding, caused by the presence of the other cell populations, can be minimized.

Separation by buoyant density can be carried out using either a continuous or a discontinuous gradient. In continuous gradients, the density of the solution increases continuously from the top to the bottom of the gradient, while discontinuous gradients consist of distinct layers of different densities. During centrifugation, the cells, irrespective of their initial position in the gradient (top, middle or bottom), migrate to a point where the density of the medium is equal to the density of the cells, or in discontinuous gradients, they reach a layer which is denser (or lighter) than the cells, at which point they stop migrating. These methods result in the cells banding according to their buoyant densities, the distance between the bands (their resolution) depending upon the quantitative difference in their buoyant densities and the density profile of the gradient. The osmolality of the gradient solution is important in determining the buoyant density of osmotically sensitive particles such as cells and other membrane bound particles. In conditions that are isoosmotic with the in vivo environment of the cells, they will retain their natural volume and buoyant densities. Variations in the osmolality will result in changes in cell volume and thus density.

In one aspect, the invention provides a method of preparing a population of substantially purified EC cells. The method includes obtaining a mixed population of cells from a subject. The cell population contains EC cells. The cell population is separated by a separation technique based on density of the cells, size of the cells, or both, as set forth above. The resulting separated population contains EC cells that are separated from other cells of the population. Additionally, the method can include removing the EC cells from the separated population to obtain substantially purified EC cells.

Also provided is a method of isolating an individual EC cell. The method includes contacting a population of cells containing EC cells with a reagent that specifically binds EC cells. The presence of an EC cell in the population is then detected and the detected EC cell is isolated.

Detecting an EC cell refers to identifying an EC cell in a population of cells containing EC cells and other cell types. Such detecting can be achieved by various methods including immunocytochemical methods, histochemical methods and in situ hybridization. Immunocytochemical methods are well known in the art. Immunocytochemical methods include labeling the cell using a primary antibody, a monoclonal or polyclonal antibody that specifically binds an antigen contained in or on an EC cell. The primary antibody is then detected by using a secondary antibody, which is an antibody that specifically recognizes the primary antibody, and which carries a detectable marker. Markers include, but are not limited to, fluorescent molecules such as fluoroscein isothiocyanate (FITC), which can be detected by routine microscopy techniques, or biotin, which can be detected by a biotin/avidin labeling system. To detect EC cells, an antibody that specifically binds a hormone synthesized or secreted by an EC cell can be used. For example, antibodies that specifically bind serotonin, histamine, chromagranin A, VMAT 1and 2 (vesicular monoamine transporter 1 and 2) or guanylin would detect EC cells. Similarly antibodies that specifically bind synthetic enzymes used in the biosynthesis of the hormones (e.g. serotonin and histamine) can be used to detect EC cells, for example, tryptophan hydroxylase or histamine decarboxylase. Furthermore, antibodies that that specifically bind EC cell surface receptors, such as CCK-A and CCK-B (cholecystokinin A and B) receptors, can also be used to detect EC cells.

As used herein, the term “specific interaction” or “specifically binds” or the like means that two molecules form a complex that is relatively stable under physiologic conditions. The term is used herein in reference to various interactions, including, for example, the interaction of an antibody and an antigen in an EC cell, the interaction of a probe and nucleic acids of an EC cell, and the like. A specific interaction can be characterized by a dissociation constant of at least about 1×10⁻⁶ M, generally at least about 1×10⁻⁷ M, usually at least about 1×10⁻⁸ M, and particularly at least about 1×10⁻⁹ M or 1×10⁻¹⁰ M or greater. A specific interaction generally is stable under physiological conditions, including, for example, conditions that occur in a living individual such as a human or other vertebrate or invertebrate, as well as conditions that occur in a cell culture such as used for maintaining mammalian cells or cells from another vertebrate organism or an invertebrate organism. Methods for determining whether two molecules interact specifically are well known in the art.

Further methods of detecting EC cells include in situ hybridization. Methods of in situ hybridization are well known to those of skill in the art. The method includes labeling the cell with an oligonucleotide that specifically binds a nucleic acid sequence encoding tryptophan hydroxylase, guanylin or monamine transporter located in EC cells.

In general, the polynucleotides used in the practice of the present invention will have a sequence that is complementary to at least a portion of the target polynucleotide. Absolute complementarity is not required. Thus, reference herein to a “nucleotide sequence complementary to” a target polynucleotide does not necessarily mean a sequence having 100% complementarity with the target segment. In general, any polynucleotide having sufficient complementarity to form a stable duplex with the target, a polynucleotide that is hybridizable under stringent conditions, is suitable. Stable duplex formation depends on the sequence and length of the hybridizing polynucleotide and the degree of complementarity with the target polynucleotide. Generally, the larger the hybridizing polymer (or oligomer), the more mismatches can be tolerated. One skilled in the art can readily determine the degree of mismatching which can be tolerated between any given probe oligomer and the target sequence, based upon the melting point, and therefore the thermal stability, of the resulting duplex. The thermal stability of hybrids formed by probe (antisense) polynucleotides are determined by way of melting, or strand dissociation, curves. The temperature of fifty percent strand dissociation is taken as the melting temperature, Tₘ, which, in turn, provides a convenient measure of stability. Tₘ measurements are typically carried out in a saline solution at neutral pH with target and antisense polynucleotide concentrations at between about 1.0-2.0 μM. Typical conditions are as follows: 150 mM NaCl and 10 mM MgCl₂ in a 10 mM sodium phosphate buffer (pH 7.0) or in a 10 mM Tris-HCl buffer (pH 7.0). Data for melting curves are accumulated by heating a sample of the antisense polynucleotide/target polynucleotide complex from room temperature to about 85-90° C. As the temperature of the sample increases, absorbance light at 260 nm is monitored at 1° C. intervals, e.g., using a spectrophotometer and temperature controller, or like instruments. Such techniques provide a convenient means for measuring and comparing the binding strengths of polynucleotides of different lengths and compositions.

In nucleic acid hybridization reactions, the conditions used to achieve a particular level of stringency will vary, depending on the nature of the nucleic acids being hybridized. For example, the length, degree of complementarity, nucleotide sequence composition (e.g., GC v. AT content), and nucleic acid type (e.g., RNA v. DNA) of the hybridizing regions of the nucleic acids can be considered in selecting hybridization conditions. An additional consideration is whether one of the nucleic acids is immobilized, for example, on a filter.

An example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42° C. (moderate stringency conditions); and 0.1×SSC at about 68° C. (high stringency conditions). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically.

In another embodiment, the EC cells are isolated by laser capture microscopy. Human tissues are composed of complex admixtures of different cell types and their biologically meaningful analysis necessitates the procurement of pure samples of the cells of interest. A recent advance in microdissection techniques is laser capture microdissection (LCM) which provides a reliable method to procure pure populations of cells from specific microscopic regions of tissue sections in one step under direct visualization. The EC cells are identified using techniques known to those of skill in the art, for example, by labeling with specific antibodies. The cells of interest are transferred to a polymer film activated by laser pulses. The exact morphology of the procured cells (with intact DNA, RNA and proteins) is retained and held on the transfer film. Using LCM, cDNA libraries can be developed from pure cells obtained directly from stained tissue, and microhybridization arrays of thousands of genes can be used to examine gene expression in microdissected human tissue biopsies. The fluctuation of expressed genes or alterations in the cellular DNA that correlate with a particular disease stage can be compared within or between individual patients. Such a fingerprint of gene-expression patterns can provide crucial clues for etiology to contribute to diagnostic decisions and therapies tailored to the individual subject. (Simone et al., Trends in Genetics, 14:272-276, 1998.)

Also provided by the invention is a method of identifying a polynucleotide that is associated with an EC cell-associated disorder in EC cells. The method comprises contacting an EC cell from a subject with an EC cell-associated disorder with an array of probes representative of EC cell nucleic acid molecules expressed in EC cells from a subject having an EC cell-associated disorder. The expression of nucleic acid molecules in the EC cell is then detected. By comparing the expression of nucleic acid molecules in the cell to the expression of an EC cell from a normal subject, i.e. a subject without an EC cell-associated disorder, a polynucleotide associated with abnormal neuroendocrine hormone expression in EC cell can be identified. The expression of an EC cell from a normal subject can be obtained by contacting an EC cell from a subject without an EC cell-associated disorder with an array of probes or by using a known expression profile. Additionally, by creating a panel of profiles representative of various disorders, an otherwise unidentified disorder of EC cells can be identified simply by comparing the unknown profile with the known profiles and determining which known profile matches the unknown profile. In one embodiment, the comparison is automated.

As used herein, “an EC cell-associated disorder” means any disorder, or disease that is related to EC cells. In one embodiment, the EC cell-associated disorder is abnormal neuroendocrine hormone expression. Additional exemplary disorders include, but are not limited to, carcinoid tumors, carcinoid syndrome, and irritable bowel syndrome. The EC cells can be gastric EC cells, intestinal EC cells, colorectal EC cells, or appendical EC cells.

As used herein, “abnormal neuroendocrine hormone expression” refers to a level or amount of expression in EC cells of any one a number of hormones, wherein the level or amount of expression is different from the level or amount of expression normally observed in an otherwise healthy individual. Exemplary hormones include, but are not limited to serotonin, histamine, VIP, glucagon, somatostatin, neurotensin, guanylin and others. The level or amount of neuroendocrine hormones can readily be assessed by methods known to those of skill in the art, for example, by radioimmunoassay, enzyme-linked immunosorbant assay, high pressure liquid chromatography, radiolabelling methods, and the like.

As used herein, the term “abnormal,” when used in reference to the amount, development or metabolic activity of an EC cell, is used in a relative sense in comparison to an amount, development or metabolic activity that a skilled clinician or other relevant artisan would recognize as being normal or ideal. Such normal or ideal values are known to the clinician and are based on average values generally observed or desired in an EC cell from a healthy subject in a corresponding population.

The contacting is performed under conditions that allow for specific hybridization of a nucleic acid molecule with probe having sufficient complementarity, for example, under stringent hybridization conditions.

As used herein, the term “array of probes representative of EC cell nucleic acid molecules” means an organized group of oligonucleotide probes that are linked to a solid support, for example, a microchip such as a silicon wafer; a glass slide; or a bead, wherein the probes can hybridize specifically and selectively to nucleic acid molecules expressed in EC cells. Microarray technology is one approach to comparatively analyze genome-wide patterns of mRNA expression. For mRNA expression studies, a goal is to develop arrays which contain every gene in a genome or which contain every gene expressed in a cell type, for example EC cells (both from a subject not having an EC cell-associated disorder, and from a subject having an EC cell-associated disorder) against which mRNA expression levels can be quantitatively assessed. However, in alternate applications, arrays can be used where the oligonucleotide probes are specific only for genes present in certain disorders or are specific only for a normal cell.

In general, an array of probes that is “representative” of EC cells will identify at least about 30% or the expressed nucleic acid molecules in an EC cell, generally at least about 50% or 70%, particularly at least about 80% or 90%, and in another embodiment will identify all of the expressed nucleic acid molecules.

The technology for cDNA based microarray technology is based on an approach where cDNA clone inserts are printed, deposited or immobilized onto a glass slide, silicon wafer or the like, in discrete locations, which can be addressable positions in an array. The inserts are subsequently hybridized to two differentially labeled probes, e.g. fluorescently differentially labeled probes. The probes are pools of cDNAs that are generated after isolating mRNA from cells or tissues in two states that are to be compared, e.g., EC cells obtained from a subject having or suspected of having an EC cell-associated disorder compared to EC cells obtained from a subject not having an EC cell-associated disorder.

In one embodiment, a plurality of probes representative of EC cell nucleic acid molecules expressed in EC cells is provided, where the probes of the plurality can be deposited on the solid support such that individual populations of probes of the plurality are substantially isolated from each other. In addition, the samples generally are arranged in an array or other reproducible pattern, such that each sample can be assigned an address (i.e., a position on the array), thus allowing a convenient means to identify a polynucleotide associated with an EC cell-associated disorder identifying a signal, phenotype, or the like at the particular position. An additional advantage of printing probes of a plurality in an array, particularly an addressable array, is that an automated system can be used for adding or removing reagents from one or more of the positions at various times, or for adding different reagents to particular positions. In addition to the convenience of examining multiple samples at the same time, such high throughput assays provide a means for examining duplicate, triplicate, or more aliquots of a single sample, thus increasing the validity of the results obtained, and for examining control samples under the same conditions as the test samples, thus providing an internal standard for comparing results from different assays.

An array is an orderly arrangement of samples. It provides a medium for matching known and unknown DNA samples based on base-pairing rules and automating the process of identifying the unknowns. An array experiment can make use of common assay systems such as microplates or standard blotting membranes, and can be created by hand or make use of robotics to deposit the sample. In general, arrays are described as macroarrays or microarrays, the difference being the size of the sample spots. Macroarrays contain sample spot sizes of about 300 microns (micrometers) or larger and can be easily imaged by existing gel and blot scanners. The sample spot sizes in microarray are typically less than 200 microns in diameter and these arrays usually contain thousands of spots. Microarrays require specialized robotics and imaging equipment that generally are not commercially available as a complete system.

DNA microarray, or DNA chips are fabricated by high-speed robotics, generally on glass but sometimes on nylon substrates, for which probes with known identity are used to determine complementary binding, thus allowing massively parallel gene expression and gene discovery studies. An experiment with a single DNA chip can provide researchers information on thousands of genes simultaneously. A “probe” is the tethered nucleic acid with known sequence. A “target” is the free nucleic acid sample whose identity/abundance is being detected.

An array of probes can include, at two or more positions on the solid support, probes that are the same or different. Where probes at two or more positions are the same, each position can be contacted with different cell types or with similar cell types having different phenotypes, thus allowing a means to identify a differential effect of polynucleotides in different cells. For example, cells that can be contacted at two or more positions of an array that contain the same probes can be normal cells (i.e., cells from a healthy individual such as a human) and corresponding abnormal cells (i.e., the same type of cells as the normal cells, but obtained from an individual suffering from an EC cell-associated disorder), wherein the invention can allow identification of polynucleotides that differentially affect the phenotype of the abnormal cells but not the corresponding normal cells (or vice versa). In comparison, where probes at two or more positions are different, each position can, but need not, be contacted with the same cell types, which can be normal or abnormal cells as exemplified above, thus providing a means to identify polynucleotides that silence one or more genes of interest.

There are two major applications for the DNA microarray technology: (a) identification of nucleic acid sequences including genes (introns and exons, protein coding regions and regulatory regions), mutations associated with genes, and single nucleotide polymorphisms (SNPs); and (b) determination of expression level (abundance) of genes. In application type (a), probe cDNA (about 500 to 5,000 bases long) is immobilized to a solid surface such as glass using robot spotting and exposed to a set of targets either separately or in a mixture. (See Ekins and Chu, Trends in Biotechnology, 17, 217-218, 1999). In application type (b), an array of oligonucleotide (nucleic acids about 20 to about 80 base pairs in length) or peptide nucleic acid (PNA) probes is synthesized either in situ (on-chip) or by conventional synthesis followed by on-chip immobilization. The array is exposed to labeled sample DNA, hybridized, and the identity/abundance of complementary sequences are determined. Such DNA chips are commercially available, for example, from Affymetrix, Inc., which sells its photolithographically fabricated products under the GeneChip® DNA chip trademark. Many companies are manufacturing oligonucleotide-based chips using alternative in situ synthesis or depositioning technologies.

Applications (a) and (b) as described above can be used to identify gene sequences that are expressed in different amounts in a subject having or suspected of having an EC cell-associated disorder as compared to a subject not having the disorder. Exemplary sequences include hormones secreted by EC cells, EC cell receptors, EC cells regulatory molecules, e.g., molecules that stimulate secretion, or inhibit secretion, and those that affect growth.

In another embodiment of the invention, applications (a) and (b) above are used to identify specific gene sequences associated with proliferation of EC cells such as genes encoding gastrin or CCK receptors, followed by hierarchical clustering to differentiate between non-proliferating and proliferating cells. “Clustering,” as used herein refers to data analysis by grouping or segmenting data. Clustering may be hierarchical clustering or k-means clustering. Hierarchical clustering is a process of sorting the data into related groups. specifically, clustering is applied to data generated (gene intensity, and presence or absence of a gene) using the weighted pair group method with centroid average as implemented in the program Cluster 2.02 (M. Eisen, see the world wide web at URL http://www.microarrays.org, “software” link; Eisen et al., PNAS 95:14863, 1998.). The distance matrixes used are Pearson correlation for clustering the arrays and the inner products normalized to magnitude 1 for the genes. The subsequent results are analyzed using TreeView 1.45 (M. Eisen, see the world wide web at URL http://www.microarrays.org, “software” link; Eisen et al., PNAS 95:14863, 1998.). This generates cluster trees, from which specific gene families (or gene groupings) that are over- or under-expressed following a proliferative stimulus (e.g. gastrin), can be identified.

The invention further relates to a method of treating or preventing an EC cell-associated disorder. The method includes administering to a subject in need thereof, i.e. a subject having or suspected of having an EC cell-associated disorder, an effective amount of an agent that modulates activity or expression of a polynucleotide identified using array analysis. As used herein, the term “modulate the activity or expression,” when used in reference to EC cells, means that the natural expression of nucleotide sequence is increased or decreased. The expression of a hormone can be modulated by affecting a regulatory element in the gene sequence. In particular, expression can be increased or decreased with respect to the basal activity of the promoter, i.e., the level of expression, if any, in the absence of a EC cell-associated disorder; or can be increased or decreased with respect to the level of expression in the presence of the disorder. As such, an agent can act to modulate the cellular response to the disorder.

If a regulatory element is involved, such a method can be performed, for example, by contacting the regulatory element with an agent suspected of having the ability to modulate the activity of the regulatory element, and detecting a change in the activity of the regulatory element. In one embodiment, the regulatory element can be operatively linked to a heterologous polynucleotide encoding a reporter molecule, and an agent that modulates the activity of the disorder-inducible regulatory element can be identified by detecting a change in expression of the reporter molecule due to contacting the regulatory element with the agent. Such a method can be performed in vitro in an animal-free EC cell system.

The agent can decrease or increase expression directly. If a decrease in expression of a nucleic acid is desired, oligomeric antisense compounds, particularly oligonucleotides, can be used to modulate nucleic acid molecules encoding a particular polynucleotide, ultimately modulating the amount of protein product produced. This is accomplished by providing antisense compounds that specifically hybridize with one or more nucleic acids encoding the target polynucleotide. As used herein, the terms “target polynucleotide” encompass DNA, RNA (including pre-mRNA and mRNA) transcribed from such DNA, and also cDNA derived from such RNA. The specific hybridization of an oligomeric compound with its target nucleic acid interferes with the normal function of the nucleic acid. This modulation of function of a target nucleic acid by compounds that specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity which can be engaged in or facilitated by the RNA. The overall effect of such interference with target nucleic acid function is modulation of the expression of a specific gene. In the context of the present invention, “modulation” means either an increase (stimulation) or a decrease (inhibition) in the expression of a gene. In one embodiment of the present invention, inhibition is the form of modulation of gene expression and mRNA is a target.

Specific nucleic acids can be targeted for antisense. Targeting an antisense compound to a particular nucleic acid, in the context of this invention, is a multi-step process. The process usually begins with the identification of a nucleic acid sequence whose function is to be modulated. This can be, for example, a cellular gene (or mRNA transcribed from the gene) whose expression is associated with a particular disorder or disease state, or a nucleic acid molecule from an infectious agent. In the present invention, the target is a nucleic acid molecule encoding a specific surface receptor essential for growth or inhibition of growth. The targeting process can also include determination of a site or sites within the nucleic acid molecule for the antisense interaction to occur such that the desired effect, e.g., detection or modulation of expression of the protein, will result. In the present invention, such sites include, but are not limited to the regions important for transcription initiation, translation initiation, and the proximal promoter region. The translation initiation codon is typically 5′-AUG (in transcribed mRNA molecules; 5′-ATG in the corresponding DNA molecule), the translation initiation codon is also referred to as the “AUG codon,” the “start codon” or the “AUG start codon.” A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (prokaryotes). It is also known in the art that eukaryotic and prokaryotic genes can have two or more alternative start codons, any one of which can be utilized for translation initiation in a particular cell type or tissue, or under a particular set of conditions. In the present invention, “start codon” and “translation initiation codon” refer to the codon or codons that are used in vivo to initiate translation of an mRNA molecule transcribed from a gene encoding a protein, regardless of the sequence(s) of such codons. In one embodiment of the present invention, a sequence complementary to the entire cDNA is used. Such an antisense molecule can target either the mature mRNA, or the individual exons within the pre-mRNA.

It is also known in the art that a translation termination codon (or “stop codon”) of a gene can have one of three sequences, i.e., 5′-UAA, 5′-UAG and 5′-UGA (the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which is known in the art to refer to the region between the translation initiation codon and the translation termination codon, is also a region which can be targeted effectively. Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA or corresponding nucleotides on the gene and the 3′ untranslated region (3′UTR), known in the art to refer to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The 5′ cap region can also be a target region.

Although some eukaryotic mRNA transcripts are directly translated, many contain one or more regions, known as “introns,” which are excised from a pre-mRNA transcript to yield one or more mature mRNA. The remaining (and therefore translated) regions are known as “exons” and are spliced together to form a continuous mRNA sequence. mRNA splice sites, i.e., exon-exon or intron-exon junctions, can also be target regions, and are particularly useful in situations where aberrant splicing is implicated in disease, or where an overproduction of a particular mRNA splice product is implicated in disease. Aberrant fusion junctions due to rearrangements or deletions are also targets. Targeting particular exons in alternatively spliced mRNAs can also be effective. It has also been found that introns can also be effective target regions for antisense compounds targeted, for example, to DNA or pre-mRNA.

As used herein, “treating” or “ameliorating” refers to inhibiting or arresting the development of a disease, disorder or condition and/or causing the reduction, remission, or regression of a disease, disorder or condition. Those of skill in the art will understand that various methodologies and assays can be used to assess the development of a disease, disorder or condition, and similarly, various methodologies and assays can be used to assess the reduction, remission or regression of a disease, disorder or condition.

As employed herein, the phrase “an effective amount,” when used in reference to invention methods employing compounds identified by invention methods and pharmaceutically acceptable salts thereof, refers to a dose of compound sufficient to provide concentrations high enough to impart an effect on the recipient thereof. In one embodiment the effective amount is a “therapeutically effective amount,” which refers to a dose high enough to impart a beneficial effect on the recipient. As used herein, “an inhibitory effective amount” refers to a dose of invention compounds sufficient to provide concentrations high enough to inhibit proliferation of tumor cells. The specific effective dose level for any particular patient will depend upon a variety of factors including the disorder being treated, the severity of the disorder, the activity of the specific compound used, the route of administration, the rate of clearance of the specific compound, the duration of treatment, the drugs used in combination or coincident with the specific compound, the age, body weight, sex, diet and general health of the patient, and like factors well known in the medical arts and sciences. Dosage levels typically fall in the range of about 0.001 to 100 mg/kg/day, more specifically, about 0.05 to 10 mg/kg/day.

The terms “polynucleotide” and “oligonucleotide” refer to molecules formed from joined nucleotides. The terms “polynucleotide” (and “oligonucleotide”) include naturally occurring polynucleotides or synthetic polynucleotides formed from naturally occurring subunits or analogous subunits designed to confer special properties on the polynucleotide so that it is more stable in biological systems or binds more tightly to target sequences. It also includes modifications of the polynucleotides such as chemically linking them to other compounds that will enhance delivery to cells or to the nucleus and other compartments of cells. Further, polynucleotides of the invention can contain modified internucleotide linkages to provide stability against nucleases. For example, the invention can include phosphorothioate oligodeoxyribonucleotides. Thus, the term “polynucleotide” includes unmodified multimers of ribonucleotides and/or deoxyribonucleotides, as well as multimers wherein one or more purine or pyrimidine moieties, sugar moieties or internucleotide linkages is chemically modified.

The terms “polynucleotide” and “oligonucleotide” as used herein include polymers of ribonucleic acid (RNA) or deoxyribonucleic acid (DNA) or mimetics thereof. They also include multimers of natural or modified monomers or linkages capable of specifically binding to a target polynucleotide by way of a regular pattern of monomer-to-monomer interactions, such as Watson-Crick type of base pairing, Hoogsteen or reverse Hoogsteen types of base pairing, or the like. Further, “polynucleotide” and “oligonucleotide” include polynucleotides and oligonucleotides composed of naturally occurring nucleic acid bases, sugars and covalent internucleoside (backbone) linkages as well as polynucleotides and oligonucleotides having non-naturally-occurring portions which function similarly. Such modified or substituted polynucleotides and oligonucleotides are often preferred over native forms because of desirable properties such as, for example, enhanced cellular uptake, enhanced affinity for nucleic acid target and increased stability in the presence of nucleases.

While antisense polynucleotides and oligonucleotides are one form of antisense compound, the present invention comprehends other polymeric and oligomeric antisense compounds, including but not limited to oligonucleotide mimetics such as those described below. In one embodiment, the antisense compounds in accordance with this invention comprise from about 100 to about 3000 nucleotides. As is known in the art, a nucleoside is a base-sugar combination. The base portion of the nucleoside is normally a heterocyclic base, a purine or a pyrimidine. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to form a linear polymeric compound. The respective ends of this linear polymeric structure can be joined to form a circular structure or can remain as an open linear structure. Within the oligonucleotide structure, the phosphate groups are commonly referred to as forming the intemucleoside backbone of the oligonucleotide. The normal linkage or backbone of RNA and DNA is a 3′ to 5′ phosphodiester linkage.

Polynucleotides used in the invention methods can be modified to increase stability and prevent intracellular and extracellular degradation. In one embodiment the polynucleotides of the invention are modified to increase their affinity for target sequences, and to increase their transport to the appropriate cells and cell compartments when they are delivered into a mammal in a pharmaceutically active form.

Recombinant nucleic acid molecules comprising a polynucleotide complementary to the nucleotide sequence encoding an EC cell specific sequence include vectors containing antisense nucleic acid, ribozymes, or triplex agents to block transcription or translation of a mRNA, either by masking that mRNA with an antisense nucleic acid or triplex agent, or by cleaving it with a ribozyme in disorders associated with increased expression.

Use of a polynucleotide to stall transcription is known as the triplex strategy since the oligomer winds around double-helical DNA, forming a three-strand helix. Therefore, these triplex compounds can be designed to recognize a unique site on a chosen gene (Maher et al., Antisense Res. and Dev., 1:227, 1991; Helene, Anticancer Drug Design, 6:569, 1991).

Ribozymes are RNA molecules possessing the ability to specifically cleave other single-stranded RNA in a manner analogous to DNA restriction endonucleases. Through the modification of nucleotide sequences that encode these RNAs, it is possible to engineer molecules that recognize specific nucleotide sequences in an RNA molecule and cleave it (Cech, J. Amer. Med. Assn., 260:3030, 1988). A major advantage of this approach is that, because they are sequence-specific, only mRNAs with particular sequences are inactivated.

There are two basic types of ribozymes namely, tetrahymena-type (Hasselhoff, Nature, 334:585, 1988) and “hammerhead”-type. Tetrahymena-type ribozymes recognize sequences that are four bases in length, while hammerhead-type ribozymes recognize base sequences 11-18 in length. The longer the recognition sequence, the greater the likelihood that the sequence will occur exclusively in the target mRNA species. Consequently, hammerhead-type ribozymes are preferable to tetrahymena-type ribozymes for inactivating a specific mRNA species and 18-based recognition sequences are preferable to shorter recognition sequences.

For therapeutic or prophylactic treatment, polynucleotides are administered in accordance with this invention. Polynucleotides can be formulated in a composition, which can include carriers, thickeners, diluents, buffers, preservatives, surface active agents and the like in addition to the polynucleotide. In one embodiment, the composition is a pharmaceutical composition. The polynucleotide can be administered in conjunction with other therapeutics found effective to inhibit or prevent proliferation of tumor cells.

The antisense compounds of the invention encompass any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents. The term “prodrug” indicates a therapeutic agent that is prepared in an inactive form that is converted to an active form (i.e., drug) within the body or cells thereof by the action of endogenous enzymes or other chemicals and/or conditions. In one embodiment, prodrug versions of the oligonucleotides of the invention can be prepared as (S-acetyl-2-thioethyl) phosphate (“SATE”) derivatives according to the methods disclosed in WO 93/24510 to Gosselin et al., published Dec. 9, 1993 or in WO 94/26764 to Imbach et al.

The term “pharmaceutically acceptable salts” refers to physiologically and pharmaceutically acceptable salts of the compounds of the invention: i.e., salts that retain the desired biological activity of the parent compound and do not impart undesired toxicological effects thereto. Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. Pharm. Sci., 66:1, 1977). The base addition salts of the acidic compounds are prepared by contacting the free acid form with a sufficient amount of the desired base to produce the salt in the conventional manner. The free acid form can be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic or inorganic acid salts of the amines. Acid salts include, but are not limited to, the hydrochlorides, acetates, salicylates, nitrates and phosphates. Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo- or phospho- acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 20 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds can also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible as suitable pharmaceutically acceptable cations.

For polynucleotides, examples of pharmaceutically acceptable salts include, but are not limited, to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid, and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine, and the like.

The present invention also includes compositions and formulations. In one embodiment the compositions and formulations are pharmaceutical compositions and formulations. The compositions of the present invention can be administered in a number of ways depending upon whether local or systemic treatment is desired and upon the area to be treated. Administration can be topical (including ophthalmic, vaginal, rectal, intranasal, transdermal), oral or parenteral. Parenteral administration includes intravenous drip, continuous infusion, subcutaneous, intraperitoneal or intramuscular injection, pulmonary administration, e.g., by inhalation or intrathecal or intraventricular administration. For oral administration, it has been found that oligonucleotides with at least one 2′-substituted ribonucleotide are particularly useful because of their absorption and distribution characteristics. Oligonucleotides with at least one 2′-methoxyethyl modification are believed to be particularly useful for oral administration.

Pharmaceutical compositions and formulations for topical administration can include transdermal patches, ointments, lotions, creams, gels, drops, suppositories, sprays, liquids and powders. Conventional pharmaceutical carriers, aqueous, powder or oily bases, thickeners and the like can be necessary or desirable. Coated condoms, gloves and the like can also be useful.

Compositions and formulations for oral administration include powders or granules, suspensions or solutions in water or non-aqueous media, capsules, sachets or tablets. Thickeners, flavoring agents, diluents, emulsifiers, dispersing aids or binders can be desirable.

Compositions and formulations for parenteral, intrathecal or intraventricular administration can include sterile aqueous solutions, which can also contain buffers, diluents and other suitable additives such as, but not limited to, penetration enhancers, carrier compounds and other pharmaceutically acceptable carriers or excipients.

Compositions and/or pharmaceutical compositions or formulations comprising the oligonucleotides of the present invention can also include penetration enhancers in order to enhance the alimentary delivery of the oligonucleotides. Penetration enhancers can be classified as belonging to one of five broad categories, i.e., fatty acids, bile salts, chelating agents, surfactants and non-surfactants (Lee et al., Critical Reviews in Therapeutic Drug Carrier Systems, 8, 91, 1991; Muranishi, Critical Reviews in Therapeutic Drug Carrier Systems, 7, 1, 1990.). One or more penetration enhancers from one or more of these broad categories can be included. Penetration enhancers are described in U.S. Pat. No. 6,083,923, which is herein incorporated by reference.

Various fatty acids and their derivatives which act as penetration enhancers include, for example, oleic acid, lauric acid, capric acid, myristic acid, palmitic acid, stearic acid, linoleic acid, linolenic acid, dicaprate, tricaprate, recinleate, monoolein (1-monooleoyl-rac-glycerol), dilaurin, caprylic acid, arachidonic acid, glyceryl 1-monocaprate, 1-dodecylazacycloheptan-2-one, acylcarnitines, acylcholines, mono- and di-glycerides and physiologically acceptable salts thereof (i.e., oleate, laurate, caprate, myristate, palmitate, stearate, linoleate, etc.) (Lee et al., Critical Reviews in Therapeutic Drug Carrier Systems, page 92, 1991; Muranishi, Critical Reviews in Therapeutic Drug Carrier Systems, 7, 1, 1990; El-Hariri et al., J. Pharm. Pharma. 44, 651, 1992. Examples of some presently fatty acids are sodium caprate and sodium laurate, used singly or in combination at concentrations of 0.5 to 5%.

The physiological roles of bile include the facilitation of dispersion and absorption of lipids and fat-soluble vitamins (Brunton, Chapter 38 In: Goodman & Gilman's The Pharmacological Basis of Therapeutics, 9th Ed., Hardman et al., eds., McGraw-Hill, New York, N.Y., pp. 934-935, 1996.). Various natural bile salts, and their synthetic derivatives, act as penetration enhancers. Thus, the term “bile salt” includes any of the naturally occurring components of bile as well as any of their synthetic derivatives. One useful bile salt is chenodeoxycholic acid, sodium salt (CDCA) (Sigma Chemical Company, St. Louis, Mo.), generally used at concentrations of 0.5 to 2%.

Complex formulations comprising one or more penetration enhancers can be used. For example, bile salts can be used in combination with fatty acids to make complex formulations. Combinations can include CDCA combined with sodium caprate or sodium laurate (generally 0.5 to 5%).

Chelating agents include, but are not limited to, disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines) (Lee et al., Critical Reviews in Therapeutic Drug Carrier Systems, page 92, 1991; Muranishi, Critical Reviews in Therapeutic Drug Carrier Systems, 7, 1, 1990; Buur et al., J. Control Rel., 14, 1990). Chelating agents have the added advantage of also serving as DNase inhibitors.

Surfactants include, for example, sodium lauryl sulfate, polyoxyethylene-9-lauryl ether and polyoxyethylene-20-cetyl ether (Lee et al., Critical Reviews in Therapeutic Drug Carrier Systems, page 92, 1991); and perfluorochemical emulsions, such as FC-43 (Takahashi et al., J. Pharm. Pharmacol., 40:252, 1988).

Non-surfactants include, for example, unsaturated cyclic ureas, 1-alkyl- and 1-alkenylazacyclo-alkanone derivatives (Lee et al., Critical Reviews in Therapeutic Drug Carrier Systems, page 92, 1991); and non-steroidal anti-inflammatory agents such as diclofenac sodium, indomethacin and phenylbutazone (Yamashita et al., J. Pharm. Pharmacol., 39:621, 1987).

As used herein, “carrier compound” refers to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The co-administration of a nucleic acid and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor. For example, the recovery of a partially phosphorothioated oligonucleotide in hepatic tissue is reduced when it is coadministered with polyinosinic acid, dextran sulfate, polycytidic acid or 4-acetamido-4′-isothiocyano-stilbene-2,2′-disulfonic acid (Miyao et al., Antisense Res. Dev., 5, 115, 1995; Takakura et al., Antisense & Nucl. Acid Drug Dev., 6, 177, 1996.).

In contrast to a carrier compound, a “pharmaceutically acceptable carrier” (excipient) is a pharmaceutically acceptable solvent, suspending agent or any other pharmacologically inert vehicle for delivering one or more nucleic acids to an animal. The pharmaceutically acceptable carrier can be liquid or solid and is selected with the planned manner of administration in mind so as to provide for the desired bulk, consistency, etc., when combined with a nucleic acid and the other components of a given pharmaceutical composition. Typical pharmaceutically acceptable carriers include, but are not limited to, binding agents (e.g., pregelatinized maize starch, polyvinyl-pyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrates (e.g., starch, sodium starch glycolate, etc.); or wetting agents (e.g., sodium lauryl sulphate, etc.). Sustained release oral delivery systems and/or enteric coatings for orally administered dosage forms are described in U.S. Pat. Nos. 4,704,295; 4,556,552; 4,309,406; and 4,309,404.

The compositions of the present invention can additionally contain other adjunct components conventionally found in pharmaceutical compositions, at their art-established usage levels. Thus, for example, the compositions can contain additional compatible pharmaceutically-active materials such as, e.g., antipruritics, astringents, local anesthetics or anti-inflammatory agents, or can contain additional materials useful in physically formulating various dosage forms of the composition of present invention, such as dyes, flavoring agents, preservatives, antioxidants, opacifiers, thickening agents and stabilizers. However, such materials, when added, should not unduly interfere with the biological activities of the components of the compositions of the invention.

Regardless of the method by which the compositions of the invention are introduced into a patient, colloidal dispersion systems can be used as delivery vehicles to enhance the in vivo stability of the compounds and/or to target the compounds to a particular organ, tissue or cell type. Colloidal dispersion systems include, but are not limited to, macromolecule complexes, nanocapsules, microspheres, beads and lipid-based systems including oil-in-water emulsions, micelles, mixed micelles, liposomes and lipid:oligonucleotide complexes of uncharacterized structure. In one embodiment, a colloidal dispersion system is a plurality of liposomes. Liposomes are microscopic spheres having an aqueous core surrounded by one or more outer layer(s) made up of lipids arranged in a bilayer configuration (see, generally, Chonn et al., Current Op. Biotech., 6, 698, 1995.). An example of liposome preparation is described in U.S. Pat. No. 6,083,923, which is herein incorporated by reference.

Also provided is a cDNA or genomic DNA library prepared from EC cells obtained from a subject having or suspected of having a disorder associated with EC cells, wherein the EC cells exhibits altered phenotypic expression.

Also provided in the present invention is a method of preventing or inhibiting fibrosis. The method includes inhibiting the synthesis, post-translation modification, activation, packaging, delivery, or binding of a factor essential for the production of fibrosis. Preferably, the essential factor is produced in EC cells. Alternatively, the essential factor interacts with, or is synergistic with, a second factor produced by EC cells. Therefore, by administering to a subject having or suspected of having fibrosis, an agent that modulates expression of a factor essential for the production of fibrosis, fibrosis can e treated or prevented. Factors related to cytokines and collagen synthesis involved in intestinal scarring have been examined, including transforming growth factor beta (TGF-β) and connective tissue growth factor (CTGF). (Polynucleotides encoding CTGF are disclosed in U.S. Pat. No. 5,585,270.) TGF-β has been found in fibrotic lesions and is believed to be a critical profibrotic mediator due to its known ability to stimulate collagen synthesis (Ignotz, Massague, J. Biol. Chem., 261, 4337, 1986.). In one study of patients with carcinoid disease (Chaudhry et al., Anticancer Res., 14, 2085, 1994.), EC tumor cells expressed TGF-β while stromal cells expressed the receptor, which suggests that EC cells play an important role in the interaction of tumor and stromal cells. Examples 5 and 6 show that CTGF messenger RNA is present more often in tumor tissue compared to normal unaffected tissue (p<0.05), that serum CTGF is elevated in patients with ideal carcinoids compared to other tumors (p=0.02), and that CTGF correlates with serum levels of chromogranin A (a marker of EC cell tumor mass) (R=0.83, p<0.03). These data demonstrate that EC cell tumors produce CTGF, which can participate directly or indirectly in the development of these fibrotic lesions.

The process of normal wound repair after tissue injury follows a closely regulated sequence involving inflammation, the recruitment, activation and proliferation of fibroblasts and the secretion of extracellular matrix, which culminates in healing and termination of the proliferative and secretory processes. In pathological fibrosis, the normal termination and resolution stages are abrogated and fibroblast activity continues unabated causing excessive accumulation of extracellular matrix, dominated by fibrillar collagens, which results in the disruption of normal tissue architecture and function. Pathological fibrosis can occur in almost any organ or tissue in the body, including the liver, kidneys, lungs, digestive system, the skin, and the like. The cellular and molecular events underlying the progression of fibrosis in whatever tissue is affected can provide a scientific basis for the discovery and development of new treatment strategies.

The pathogenesis of fibrosis is generally known. Tissue injury is caused by a range of agents, including acute and chronic infections, diabetes, hypertension, autoimmunity, toxic chemicals, irritant dusts, trauma and the like, which initiate an inflammatory process in which cytokines are released from activated platelets, monocyte-macrophages and neutorphils. Other fibrogenic effectors, such as angiotensis II and endothelins, are released from the kidneys and endothelial cells, respectively. All these fibrogenic effectors recruit and activate fibroblasts or related cells specific to certain tissues, such as the hepatic stellate cells of the liver and the mesangial cells of the renal glomerulus. The fibroblasts and related cells secrete the extra-cellular matrix materials whose inappropriate accumulation characterizes the fibrotic lesion.

The following examples are intended to illustrate but not limit the invention.

### EXAMPLE 1

### Identification of Genes Selectively Expressed by Gastric EC Cells

Rat and human EC cells share the same ultrastructure, a large nucleus that is ovoid in shape with a regular outline, cytoplasm that contains well-developed Golgi-apparatus and rough endoplasmic reticulum, and large vacuolated granules with small electron dense granules, often regularly shaped and in large numbers (Solcia et al., International Reviews in Cytology, 42:223, 1975; Buffa et al., Cell. Tiss. Res., 192: 227, 1978). Biochemically, the same protein products are present in both human and rat EC cells (Solcia et al., International Reviews in Cytology, 42:223, 1975).

Cell preparations were obtained from male rats (200-225 g). Two preparations of total gastric mucosal cells were obtained by mucosal scrapping. Total RNA was isolated from each mucosal sample (n=2) using the RNeasy kit (Qiagen). Target cRNAs were generated and hybridized to rat RU34 GeneChip sets (Affymetrix, Santa Clara, Calif.). The intensity was similarly scaled for each chip (intensity=100) and those genes present in each sample identified using the GENECHIP software. This allowed the generation of gene transcript databases (genes expressed/present) for each of the two gastric mucosal cell samples. This data was used in subtractive analysis to generate neuroendocrine cell gene databases and then an EC database. A comparison of the databases allowed identification of genes selectively expressed in the EC database, as compared to the neuroendocrine cell gene database.

### EXAMPLE 2

### Genes Selectively Expressed by Gastric EC Cells

Genes selectively expressed by gastric EC cells were obtained by comparing gene expression of pools of fractionated neuroendocrine cells, including EC cells. Five female Sprague-Dawley rats (body wt 180-200 g; Charles River) were anesthetized by carbon dioxide and killed by cervical dislocation. Mucosal cells from the stomachs are isolated by pronase digestion (1.3 mg/ml) with an everted sac technique. The resulting crude cell preparation was fractionated by counterflow elutriation (flow rate of 20 ml/min, rotor speed 2,000 rpm) and subsequent density gradient centrifugation (1.050-1.075 g/mL gradient interface). This technique was repeated on twenty different days, generating 20 preparations. Five preparations were pooled to generate sufficient cells for RNA isolation. This resulted in 4 pools of cells. Total RNA was isolated from each cell pool (n=4) using the RNeasy kit (Qiagen). Target cRNAs were generated and hybridized to rat RU34GeneChip sets (Affymetrix, Santa Clara, Calif.). The intensity was similarly scaled for each chip (intensity=100) and those genes present in each sample identified using the GENECHIP software. This allowed the generation of gene transcript databases (genes expressed/present) for each of the four gastric neuroendocrine cell pools. An examination of these four databases using the MS Excel program demonstrated that tryptophan hydroxylase, the marker gene for serotonin biosynthesis, was present in two of the cell pools, indicating the presence of EC cells. Subtraction of the genes (MS Excel program/pivot table) expressed in the neuroendocrine pools from the two EC databases identified the following twelve genes that were present only in the EC cell:

Rat PACAP receptor (Accession no. D14909)

Rat 5-hydroxytryptamine-1a receptor (5-HT-1a) (Accession no. J05276)

Rat glutamate receptor (GluR-B) mRNA, complete cds (Accession no. M36419)

RATGLURD Rat glutamate receptor (GluR-D) (Accession no. M36421)

beta 3-adrenergic receptor {spliced version} (Accession no. S56481)

gonadotropin-releasing hormone receptor (rats, pituitary gland, mRNA, 2256 nt) (Accession no. S59525)

R. norvegicus prostaglandin F2a receptor mRNA (Accession no. U26663)

Rat mRNA for fast nerve growth factor receptor (NGFR) (Accession no. X05137)

Rat mRNA for kainate receptor subunit (ka1) (Accession no. X59996)

R. norvegicus SRL mRNA for stomach fundus serotonin receptor (Accession no. X66842)

R. norvegicus mRNA for retinoic acid X receptor gamma-1, partial (Accession no. AJ223083)

R. norvegicus gene for alpha2-C4 adrenergic receptor (Accession no. X57659)

The above identified genes therefore are expressed in gastric EC cells and are not generally expressed in other stomach or intestinal cells.

### EXAMPLE 3

### Cell Secretion by Intestinal EC Cells

Male Sprague-Dawley rats (n=5 per preparation) were euthanized and the ileum was completely removed and divided into 2-3 pieces. Following end-ligation, these pieces of intestine were everted and filled with an enzyme mix of Pronase E (0.7 mg/mL), and collagenase (0.25 mg/ml). Cells were separated from the muscularis by alternative switching between a calcium containing Respiration medium and a 2 mM EDTA-containing digestive medium (with shaking). A total of 1-2×10⁸ cells were obtained from each preparation. Five fractions of cells (F1-F5) were separated by counterflow-elutriation using flow rates of 25 ml/min at 2,500 rpm. Cells (100,000) were cultured overnight in Dulbecco's modified Eagles medium (DMEM)-F12 medium supplemented with 2 mg/ml bovine serum albumin, 5% fetal bovine serum, 5 mg/l gentamicin, 5 mg/l insulin, 5 mg/l transferring, 5 μg/l sodium selenite, and 1 nM hydrocortisone). After removal of the medium, fresh culture medium containing agonists (PACAP, galanin, forskolin, each at 10 nM) was added to the cells for 60 minutes. This medium was removed and the serotonin measured using an serotonin EIA kit and expressed as ng serotonin/100,000 cells/60 min.

The basal serotonin release was 1.25±0.04 ng serotonin/105 cells/60 min. Forskolin (an activator of adenylyl cyclase) was used as a positive control and stimulated serotonin release 2.1±0.14-fold versus controls (p<0.05). Both PACAP (10⁻⁸M; 0.88±0.06 versus controls, p<0.05) and galanin (10⁻⁸M; 0.85±0.03 versus controls, p<0.03) negatively regulated serotonin release. These data are consistent with studies conducted in guinea pig and rat intestine and in human EC tumors transplanted into the anterior eye chamber of the rat (Fujimiya et al., Am. J. Physiol. 275:G731, 1998; Racke et al., Br. J. Pharmacol. 95:923, 1988; Racke et al., Behavior Brain Res., 73:83, 1996; Nilsson et al., Cancer, 58: 676, 1986).

### EXAMPLE 4

### Generation of an Enriched EC Cell Population

An enriched EC cell preparation was generated from rat small intestine using elutriation. Modification of rotor speed and pump flow-rate allowed for the collection and elution of populations of cells characterized by specific sizes/densities. Centrifugation speeds ranged from about 2,000 to 2,500 rpm and flow rates ranged from about 12 to 100 ml/min for elutriation.

This approach allowed identification of two populations of EC-enriched cells (10±2-fold versus non-elutriated cells), sized 10-12.5 um and a second EC enriched cell population (5.7±0.3-fold versus non-elutriated cells) of 14-16 um in size. Differential elutriation resulted in fold-enrichments of 5.9-15.1 of serotonin cells compared to the starting source. Additionally, the use of Nycodenz gradient centrifugation (1.050-1.075 g/mL gradient interface) may be used on fractions to further enrich cells by density. The gradient was calculated to collect cells at the interface between 1.050-1.075 g/mL. The serotonin content of the cells obtained at this interface ranged from 44.1-48.1 ng serotonin/mg protein. Nycodenz gradient resulted in an additional 4.6-15.6-fold enrichment of the different elutriated cell fractions, fold-enrichments of 45±5-fold versus non-elutriated cells). This resulted in a 60-70% population of EC cells.

### EXAMPLE 5

### CTGF mRNA in Tumor Tissue

Total RNA was extracted from carcinoid tumor tissue and adjacent non-tumor tissue using the RNeasy protocol. Then first strand cDNA synthesis was performed using the SuperScript Gibco BRL protocol. Specific DNA amplification through PCR was carried out in a thermal cycler (MJ Research) with specific primer sets on 0.7 ug of cDNA template. It was found that more patients with carcinoids expressed CTGF mRNA (16/19; 84%) compared to normal tissue (4/9; 44%) (p<0.05).

### EXAMPLE 6

### CTGF Serum Studies

To detect plasma CTGF, a sandwich ELISA with two different anti-human CTGF mAbs (MHCT1 and MHCT2; mouse IgG1) was used. ELISA plates (96-well, Corning) were coated with 50 ml of MHCT1 mAb or isotype-matched control mAb (WT.3; anti-rat CD18) at 10 mg/ml in PBS for 1 hour at room temperature (RT). After washing, unbound sites were blocked by incubation with 200 ml of blocking reagent for 2 hours at RT. After washing three times with 0.1% Tween 20 in PBS (PBS-T), 50 ml of samples was added to the wells and incubated for 1 hour. After washing three times, 50 ml of biotinylated MHCT2 mAb at 2 mg/ml in 1% BSA-PBS-T was added. The plates were incubated for 1-h at RT and subsequently washed three times, and 50 ml of a 1/1000 dilution of streptavidin-b-D-galactosidase (Gibco BRL, Gaithersburg, Md.) is added. After a 1 hour incubation, the plates were washed three times, 50 ml of 1% 4-methyl-umbelliferyl-b-D galactoside (Sigma) is added, and the fluorescence intensity of the wells was determined after 10 min at 460 nm (excitation; 355 nm) by a Fluoroskan II microplate fluorometer (Lab Systems, Hampshire, UK).

Plasma CTGF was examined in twenty-one patients. The levels of plasma CTGF ranged from 7.2-52 ng/ml The twenty-one patients were divided into three groups: ileal carcinoids (n=13), other tumors (including two gastro-duodenal neuroendocrine tumors) (n=5) and three patients who underwent recent hernia repair. CTGF levels in patients with ileal carcinoids were significantly elevated (p=0.013) compared to patients with other tumors.

Data for serum markers (serotonin, gastrin, pancreatic polypeptide, vasoactive intestinal polypeptide (VIP) and substance P) were available for seven patients. A comparison (linear regression analysis) of this data with plasma CTGF demonstrated that there was a positive correlation between CTGF and chromogranin A levels (p=0.02, n=7).

Although the invention has been described with reference to the above, it will be understood that modifications and variations are encompassed within the spirit and scope of the invention. Accordingly, the invention is limited only by the following claims.

What is claimed is:

1. A method of preparing substantially purified enterochromaffin (EC) cells, comprising:
a) obtaining a mixed population of cells containing EC cells from a subject;
b) separating the population of cells by a separation technique based on density of the cells, size of the cells, or both, wherein a separated population is formed, the separated population comprising EC cells that are separated from other cells of the population, thereby preparing substantially purified EC cells.

2. The method of claim 1, further comprising:
c) removing the EC cells from the separated population, thereby obtaining substantially purified EC cells.

3. The method of claim 1, wherein the separation technique comprises density gradient centrifugation.

4. The method of claim 3, wherein the density gradient centrifugation comprises a continuous gradient.

5. The method of claim 3, wherein the density gradient centrifugation comprises a discontinuous gradient.

6. The method of claim 1, wherein the separation technique comprises elutriation.

7. The method of claim 1 wherein the separation technique comprises elutriation, followed by density gradient centrifugation.

8. The method of claim 1, wherein the separating step comprises fluorescence activated cell sorting.

9. A method of obtaining an isolated enterochromaffin (EC) cell, comprising:
a) contacting a population of cells containing EC cells with a reagent that specifically binds EC cells; and
b) isolating cells specifically bound by the reagent, thereby obtaining an isolated EC cell.

10. The method of claim 9, the method further comprising, before the isolating step, detecting cells specifically bound by the reagent.

11. The method of claim 10, wherein the detecting is performed using an immunocytochemical method, a histochemical method or in situ hybridization.

12. The method of claim 9, wherein the reagent comprises an antibody that specifically binds a hormone synthesized or secreted by the EC cell.

13. The method of claim 12, wherein the hormone is serotonin, histamine, chromagranin A, VMAT 1, VMAT 2 or guanylin.

14. The method of claim 9, wherein the reagent comprises an antibody that specifically binds a synthetic enzyme used in the biosynthesis of hormones by EC cells.

15. The method of claim 14, wherein the synthetic enzyme is tryptophan hydroxylase, histidine decarboxylase or other synthetic enzyme used in the biosynthesis of hormones by EC cells.

16. The method of claim 9, wherein the reagent comprises an antibody that specifically binds an EC cell surface receptor, and detecting the antibody specifically bound to the EC cell.

17. The method of claim 16, wherein the EC cell receptor comprises a CCK-A or CCK-B receptor.

18. The method of claim 9, wherein the reagent comprises an oligonucleotide that specifically binds a nucleic acid sequence encoding tryptophan hydroxylase or guanylin.

19. The method of claim 9, wherein the isolating is by laser capture microscopy.

20. A method of identifying a polynucleotide associated with an enterochromaffin (EC) cell-associated disorder, comprising:
a) contacting an EC cell from a subject having or suspected of having an EC cell-associated disorder with an array of probes representative of EC cell nucleic acid molecules expressed in EC cells from a subject having the EC cell-associated disorder; and
b) detecting expression of nucleic acid molecules in an EC cell of the subject,
wherein expression of nucleic acid molecules in the EC cell of the subject differs from the expression of nucleic acid molecules in an EC cell of a subject not having the disorder, thereby identifying a polynucleotide associated with an EC cell-associated disorder in an EC cell.

21. The method of claim 20, wherein the EC cell-associated disorder comprises abnormal neuroendocrine hormone expression of an EC cell.

22. The method of claim 21, wherein the neuroendocrine hormone expressed comprises serotonin, histamine, VIP, glucagons, somatostatin, neurotensin, guanylin or a combination thereof.

23. The method of claim 20, wherein the EC cell-associated disorder comprises a carcinoid tumor, carcinoid syndrome or irritable bowel syndrome.

24. The method of claim 20, wherein the wherein the EC cell-associated disorder comprises an EC cell proliferative disorder.

25. The method of claim 20, wherein the level of expression in an EC cell from a subject not having an EC cell-associated disorder is lower than the level of expression in EC cells from a subject having an EC cell-associated disorder.

26. The method of claim 20, wherein the EC cell comprises a gastric EC cell, intestinal EC cell, colorectal EC cell, or appendical EC cell.

27. A method of identifying a polynucleotide associated with enterochromaffin (EC) cell proliferation, comprising:
a) contacting a test EC cell with an array of probes representative of EC cell nucleic acid molecules expressed in normal EC cells;
b) detecting expression of nucleic acid molecules in the test EC cell;
c) performing a clustering analysis of the nucleic acid molecules expressed in the test EC cell; and
d) detecting a cluster comprising polynucleotides associated with proliferation of EC cells;
thereby identifying a polynucleotide associated with EC cell proliferation.

28. The method of claim 27, wherein the clustering is hierarchical clustering.

29. The method of claim 27, further comprising isolating the polynucleotide associated with EC cell proliferation.

30. An isolated polynucleotide associated with EC cell proliferation, the polynucleotide isolated by the method of claim 29.

31. A method of treating or preventing an EC cell-associated disorder, comprising administering to a subject having or suspected of having an EC cell-associated disorder an agent that modulates expression of a polynucleotide associated with an EC cell-associated disorder.

32. The method of claim 31, wherein the EC cell-associated disorder comprises abnormal neuroendocrine hormone expression of an EC cell.

33. The method of claim 31, wherein the wherein the EC cell-associated disorder comprises an EC cell proliferative disorder.

34. The method of claim 31, wherein the agent decreases expression of the polynucleotide.

35. The method of claim 31, wherein the agent increases expression of the polynucleotide.

36. A composition, comprising an antisense polynucleotide that specifically binds a mRNA encoding a polynucleotide associated with an EC cell-associated disorder, and a carrier.

37. The composition of claim 36, wherein the composition is a pharmaceutical composition.

38. A cDNA library prepared from the EC cells of claim 1.

39. The cDNA library of claim 38, wherein the EC cells are obtained from a subject having a EC cell-associated disorder and exhibit altered phenotypic expression.

40. A method of treating or preventing fibrosis, comprising administering to a subject having or suspected of having fibrosis an agent that modulates expression of a factor essential for the production of fibrosis.
